(19) United States
(12) Patent Application Publication
(10) Pub. No.: US 2026/0226058 A1
(43) Pub. Date: Aug. 6, 2026

(54) PREPARATION OF IMIDAZOPYRIDINE-2-CARBOXYLIC ACIDS

(71) Applicant: CORTEVA AGRISCIENCE LLC, INDIANAPOLIS, IN (US)

(72) Inventors: YUKO KIDA, ZIONSVILLE, IN (US); ANDREW GERARD SCHAFER, ZIONSVILLE, IN (US)

(73) Assignee: CORTEVA AGRISCIENCE LLC, INDIANAPOLIS, IN (US)

(21) Appl. No.: 19/128,579

(22) PCT Filed: Nov. 1, 2023
(86) PCT No.: PCT/US2023/078346

§ 371 Date: May 9, 2025

Related U.S. Application Data
(60) Provisional application No. 63/383,170, filed on Nov. 10, 2022.

(51) Int. Cl. C07D 487/04

(52) U.S. Cl. CPC C07D 487/04

(57) ABSTRACT

The present invention provides a method for preparing a compound of Formula D:

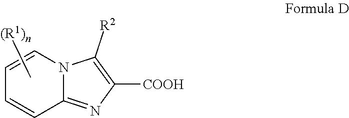

wherein each R¹ is independently halogen, nitro, SF₅, N(C₁-C₈ alkyl)(C₁-C₈ alkyl), C(═S)N(C₁-C₈ alkyl)(C₁-C₈ alkyl), SO₂N(C₁-C₈ alkyl)(C₁-C₈ alkyl), OSO₂(C₁-C₈ alkyl), OSO₂N(C₁-C₈ alkyl)(C₁-C₈ alkyl), N(C₁-C₈ alkyl)SO₂(C₁-C₈ alkyl), C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
n is 0, 1, 2, 3, or 4; and
R² is H, C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl.

### PRIORITY

This application claims priority to United States Application 63/383,170 filed Nov. 10, 2022, which is hereby incorporated herein by reference in its entirety.

### BACKGROUND

Certain nematicidal sulfonamides and methods for preparing such and their intermediates, such as imidazopyridine-2-carboxylic acids, have been previously disclosed in, for example, WO 2010/129500, WO 2012/054233, and CN108276352A. However, certain synthesis steps disclosed previously still have certain disadvantages such as elevated levels of impurities generated. Thus there remains a need for alternative ways of preparing certain nematicidal sulfonamides and their intermediates.

### SUMMARY

In one aspect, a method for preparing a compound of Formula D is provided, as follows:

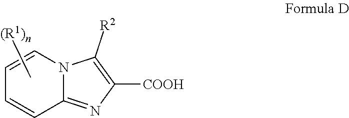

wherein each R¹ is independently halogen, nitro, SF₅, N(C₁-C₈ alkyl)(C₁-C₈ alkyl), C(═S)N(C₁-C₈ alkyl)(C₁-C₈ alkyl), SO₂N(C₁-C₈ alkyl)(C₁-C₈ alkyl), OSO₂(C₁-C₈ alkyl), OSO₂N(C₁-C₈ alkyl)(C₁-C₈ alkyl), N(C₁-C₈ alkyl)SO₂(C₁-C₈ alkyl), C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
n is 0, 1, 2, 3, or 4; and
R² is H, C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl. The method comprises (al) contacting a compound of Formula A in a solvent S1 with a compound of Formula B to form a compound of Formula C:

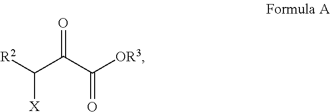

wherein X is Cl, Br, I, OSO₂Me, OSO₂(C₆H₄)CH₃, or SO₂CF₃; and R³ is C₁-C₈ alkyl or C₁-C₈ haloalkyl;

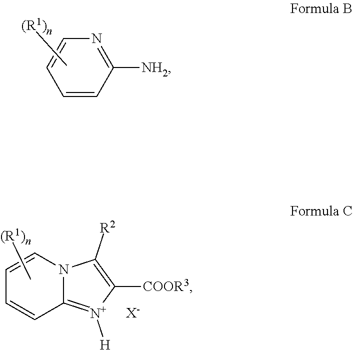

and
(b1) adding aqueous acid to the reaction mixture of Step (al).
(c1) adding base to the reaction mixture of Step (b1) to form a compound of Formula D.

In some embodiments, each R¹ is independently Cl, Br, I, C₁-C₆ alkyl, C₁-C₆ haloalkyl, C₁-C₆ alkoxy, C₁-C₆ haloalkoxy, or phenyl. In some embodiments, R² is H, C₁-C₆ alkyl, C₁-C₆ haloalkyl, C₁-C₆ alkoxy, C₁-C₆ haloalkoxy, or phenyl. In some embodiments, R² is H, CH₃, CH₂CH₃, or phenyl. In some embodiments, R² is not halogen or cyano.

In one aspect, a method of preparing a compound of Formula D:

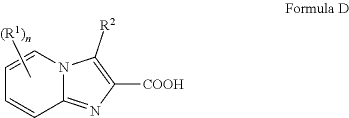

wherein each R¹ is independently halogen, nitro, SF₅, N(C₁-C₈ alkyl)(C₁-C₈ alkyl), C(═S)N(C₁-C₈ alkyl)(C₁-C₈ alkyl), SO₂N(C₁-C₈ alkyl)(C₁-C₈ alkyl), OSO₂(C₁-C₈ alkyl), OSO₂N(C₁-C₈ alkyl)(C₁-C₈ alkyl), N(C₁-C₈ alkyl)SO₂(C₁-C₈ alkyl), C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
n is 0, 1, 2, 3, or 4; and
R² is H, C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
comprising:
   (b1) adding aqueous acid to a compound of Formula C to form a reaction mixture, wherein R³ is C₁-C₈ alkyl or C₁-C₈ haloalkyl,

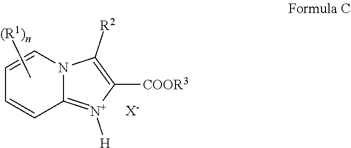

and;
(c1) adding base to the reaction mixture of Step (b1) to form a compound of Formula D.

In one aspect, a method of preparing a compound of Formula D:

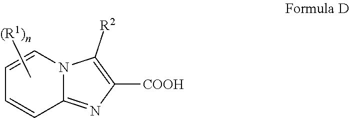

wherein each R¹ is independently halogen, nitro, SF₅, N(C₁-C₈ alkyl)(C₁-C₈ alkyl), C(═S)N(C₁-C₈ alkyl)(C₁-C₈ alkyl), SO₂N(C₁-C₈ alkyl)(C₁-C₈ alkyl), OSO₂(C₁-C₈ alkyl), OSO₂N(C₁-C₈ alkyl)(C₁-C₈ alkyl), N(C₁-C₈ alkyl)SO₂(C₁-C₈ alkyl), C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
n is 0, 1, 2, 3, or 4; and
R² is H, C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
comprising:
   (b1) adding aqueous acid to a compound of Formula C to form a reaction mixture, wherein R³ is C₁-C₈ alkyl or C₁-C₈ haloalkyl,

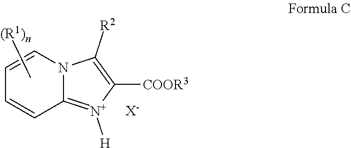

and;
(b2) adding buffer to the reaction mixture of Step (b1), and;
(c1) adding base to the reaction mixture of Step (b1) to form a compound of Formula D.

Choices of the solvent for S1 include (a) C₄-C₈ hydrocarbons (for example hexane) or C₆-C₁₀ aromatic hydrocarbons (for example, benzene, toluene, xylenes (as the pure ortho, meta and para isomers, as mixtures thereof, or as mixtures with ethylbenzene), ethyl benzene and cumene (iso-propylbenzene)), (b) halogenated benzenes (for example, chlorobenzene and 1,2-dichlorobenzene), (c) haloalkanes (for example, dichloromethane, 1,2-dichloroethane and 1-chlorobutane), (d) ethers (for example, tetrahydrofuran (THF), 2-methyltetrahydrofuran (2-Me-THF), tert-butyl methyl ether, 1,4-dioxane, and Ph₂O (diphenyl ether)); (e) esters (for example ethyl acetate, propyl acetate, and isopropyl acetate); (f) alcohols (for example methanol, ethanol, and isopropanol); and/or (g) other solvents including 1,4-diazabicyclo [2.2.2]octane (DABCO), methylene chloride (DCM), DMF, DMSO, acetonitrile (MeCN), and benzonitrile.

In some embodiments, the solvent S1 comprises C₆-C₁₀ aromatic hydrocarbons (for example, benzene, toluene, xylenes, ethyl benzene, iso-propylbenzene, and mixtures thereof). In some embodiments, the solvent 51 does not comprise acetonitrile (MeCN). In some embodiments, the solvent 51 does not comprise ether for example 1,2-dimethoxyethane. In some embodiments, the solvent S1 does not comprise tetrahydrofuran (THF).

Examples of acids suitable for use in Step (b1) include inorganic acids for example hydrochloric acid (HCl), hydrobromic acid (HBr), phosphoric acid (H₃PO₄), sulfuric acid (H₂SO₄), and boric acid (H₃BO₃); organic acids for example formic acid, acetic acid, propionic acid, benzoic acid, citric acid, malic acid, and sulfonic acids. Further examples of sulfonic acids including para-toluenesulfonic acid, methanesulfonic acid, triflic acid, and toluenesulfonic acid as a mixture of isomers. In some embodiments, the acid comprises hydrochloric acid (HCl).

Examples of bases suitable for use in Step (c1) include inorganic hydroxides, such as sodium hydroxide and potassium hydroxide, organic bases such as the alkali metal salts of alcohols, examples of which include sodium methoxide, sodium ethoxide, sodium iso-propoxide, sodium n-propoxide, potassium methoxide, potassium ethoxide, potassium 1-propoxide, and potassium 2-propoxide, and amine bases such as ammonia, monoalkylamines such as methylamine and ethylamine, dialkylamines such as dimethyl amine and triethylamine, trialkylamines such as trimethylamine and triethylamine, and aromatic amines such as pyridine. In some embodiments, the base comprises sodium hydroxide (NaOH).

In some embodiments, the invention provides a method for preparing a compound of Formula 1:

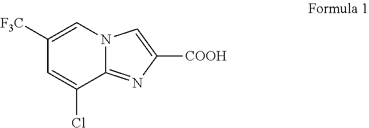 [SMILES: O=C(O)c1cn2cc(C(F)(F)F)cc(Cl)c2n1]

comprising:
(A1) contacting a compound of Formula 2 in a solvent S1 selected from benzene, toluene, xylenes, ethyl benzene, iso-propylbenzene, and mixtures thereof, with a compound of Formula 3 to form a compound of Formula 4:

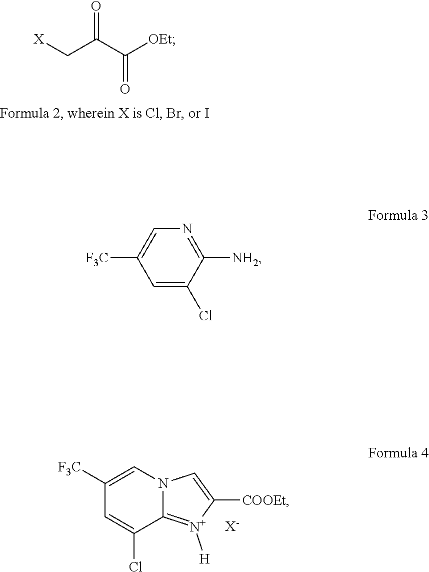

(B1) adding an aqueous acid selected from hydrochloric acid (HCl), hydrobromic acid (HBr), phosphoric acid (H₃PO₄), sulfuric acid (H₂SO₄), boric acid (H₃BO₃), or mixtures thereof, to the reaction mixture of Step (A1); and(C1) adding an aqueous base selected from ammonia, sodium hydroxide, potassium hydroxide, or mixtures thereof, to the reaction mixture of Step (B1) to generate a compound of Formula 1.

In some embodiments, the solvent S1 is selected from benzene, toluene, xylenes, and mixtures thereof. In some embodiments, the solvent S1 does not comprise acetonitrile (MeCN). In some embodiments, the solvent S1 does not comprise ether for example 1,2-dimethoxyethane. In some embodiments, the solvent S1 does not comprise tetrahydrofuran (THF).

In some embodiments, the methods provided herein reduce the amount at least one of the following impurities to less than 50 ppm:

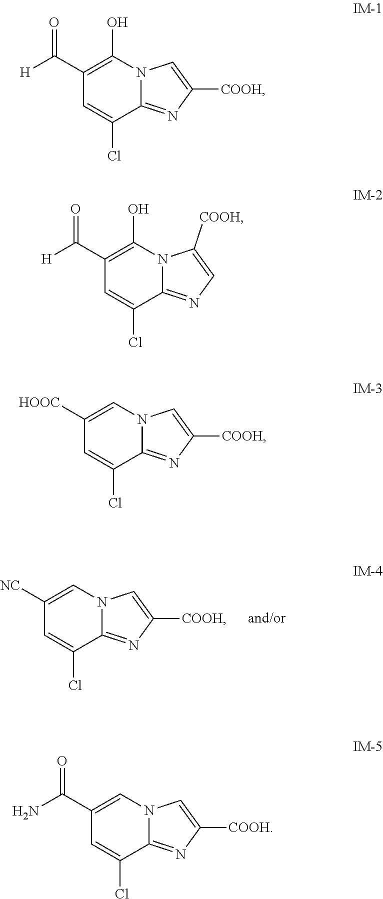 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1 ; N#Cc1cc(Cl)c2nc(C(=O)O)cn2c1 ; NC(=O)c1cc(Cl)c2nc(C(=O)O)cn2c1]

In some embodiments, the methods provided herein reduce the amount of at least one of the following impurities to less than 50 ppm:

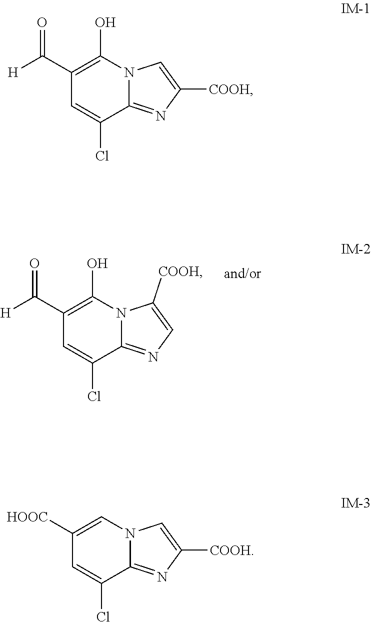 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1]

In some embodiments, the methods provided herein do not generate more than 50 ppm of any of the following impurities:

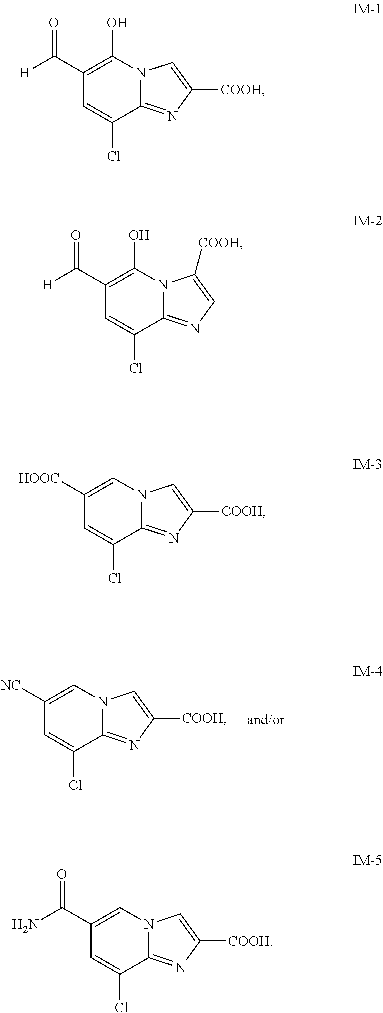 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1 ; N#Cc1cc(Cl)c2nc(C(=O)O)cn2c1 ; NC(=O)c1cc(Cl)c2nc(C(=O)O)cn2c1]

In some embodiments, the methods provided herein do not generate more than 50 ppm of any of the following impurities:

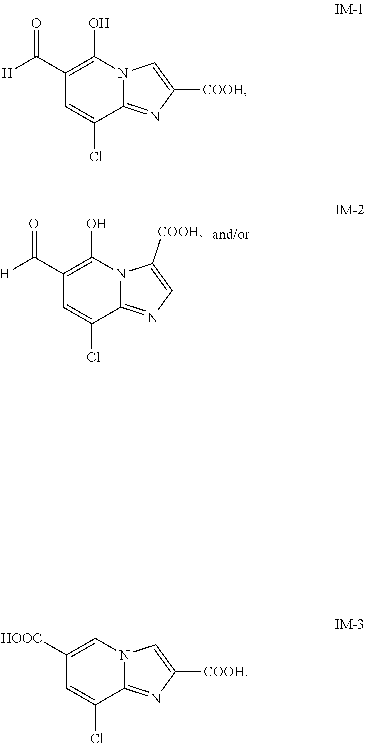 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1]

In another aspect, the present invention provides a method for preparing a compound of Formula D:

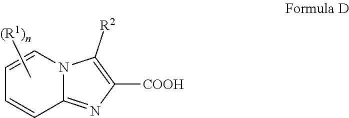

wherein each R¹ is independently halogen, nitro, SF₅, N(C₁-C₈ alkyl)(C₁-C₈ alkyl), C(═S)N(C₁-C₈ alkyl)(C₁-C₈ alkyl), SO₂N(C₁-C₈ alkyl)(C₁-C₈ alkyl), OSO₂(C₁-C₈ alkyl), OSO₂N(C₁-C₈ alkyl)(C₁-C₈ alkyl), N(C₁-C₈ alkyl)SO₂(C₁-C₈ alkyl), C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
n is 0, 1, 2, 3, or 4; and
R² is H, C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
comprising:
   (a3) contacting a compound of Formula A in a solvent Sa comprising toluene (PhCH₃) and/or acetonitrile (MeCN) with a compound of Formula B to form a compound of Formula C:

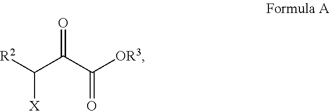

wherein X is Cl, Br, I, OSO₂Me, OSO₂(C₆H₄)CH₃, or OSO₂CF₃; and R³ is C₁-C₈ alkyl or C₁-C₈ haloalkyl;

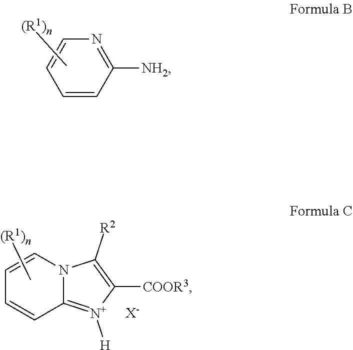

(b3) adding a base in a solvent Sβ to the reaction mixture (or isolated compounds of Formula C) of Step (a3) to generate an intermediate of Formula E:

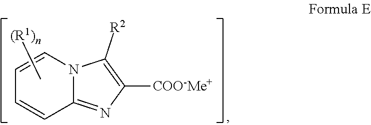

wherein M⁺ is an inorganic cation or organic cation.

In some embodiments, each R¹ is independently Cl, Br, I, C₁-C₆ alkyl, C₁-C₆ haloalkyl, C₁-C₆ alkoxy, C₁-C₆ haloalkoxy, or phenyl. In some embodiments, R² is H, C₁-C₆ alkyl, C₁-C₆ haloalkyl, C₁-C₆ alkoxy, C₁-C₆ haloalkoxy, or phenyl. In some embodiments, R² is H, CH₃, CH₂CH₃, or phenyl. In some embodiments, R² is not halogen or cyano.

Choices of the solvent for Sβ include (a) C₄-C₈ hydrocarbons (for example hexane) or C₆-C₁₀ aromatic hydrocarbons (for example, benzene, toluene, xylenes (as the pure ortho, meta and para isomers, as mixtures thereof, or as mixtures with ethylbenzene), ethyl benzene and cumene (iso-propylbenzene)), (b) halogenated benzenes (for example, chlorobenzene and 1,2-dichlorobenzene), (c) haloalkanes (for example, dichloromethane, 1,2-dichloroethane and 1-chlorobutane), (d) ethers (for example, tetrahydrofuran (THF), 2-methyltetrahydrofuran (2-Me-THF), tert-butyl methyl ether, 1,4-dioxane, and Ph₂O (diphenyl ether)); (e) esters (for example ethyl acetate, propyl acetate, and isopropyl acetate); (f) alcohols (for example methanol, ethanol, and isopropanol); and/or (g) other solvents including 1,4-diazabicyclo [2.2.2]octane (DABCO), methylene chloride (DCM), DMF, DMSO, acetonitrile (MeCN), and benzonitrile.

In some embodiments, the solvent Sβ comprises water, methanol, ethanol, isopropanol, and mixtures thereof. In some embodiments, the solvent Sα and/or the solvent Sβ do not comprise ether for example 1,2-dimethoxyethane. In some embodiments, the solvent Sα and/or the solvent Sβ do not comprise tetrahydrofuran (THF).

Examples of bases suitable for use in Step (b3) include inorganic hydroxides, such as sodium hydroxide and potassium hydroxide, organic bases such as the alkali metal salts of alcohols, examples of which include sodium methoxide, sodium ethoxide, sodium iso-propoxide, sodium n-propoxide, potassium methoxide, potassium ethoxide, potassium 1-propoxide, and potassium 2-propoxide, and amine bases such as ammonia, monoalkylamines such as methylamine and ethylamine, dialkylamines such as dimethyl amine and triethylamine, trialkylamines such as trimethylamine and triethylamine, and aromatic amines such as pyridine. In some embodiments, the base comprises sodium hydroxide (NaOH).

In some embodiments, M⁺ is an inorganic cation selected from sodium, potassium, ammonium, lithium, and mixtures thereof. In some embodiments, M⁺ is sodium. In some embodiments, M⁺ is an organic cation selected from trimethylammonium, triethylammonium, tri-n-propylammonium, triisopropylammonium, and tributylammonium.

In some embodiments, the method provided further comprises Step (c3): (c3) adding an acid to the reaction mixture of Step (b3) to generate a compound of Formula D.

Examples of acids suitable for use in Step (c3) include inorganic acids for example hydrochloric acid (HCl), hydrobromic acid (HBr), phosphoric acid (H₃PO₄), sulfuric acid (H₂SO₄), and boric acid (H₃BO₃); organic acids for example formic acid, acetic acid, propionic acid, benzoic acid, citric acid, malic acid, and sulfonic acids. Further examples of sulfonic acids including para-toluenesulfonic acid, methanesulfonic acid, triflic acid, and toluenesulfonic acid as a mixture of isomers. In some embodiments, the acid comprises hydrochloric acid (HCl).

In some embodiments, the invention provides a method for preparing a compound of Formula 1:

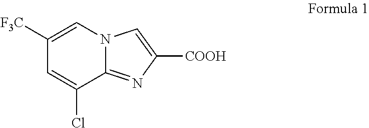 [SMILES: O=C(O)c1cn2cc(C(F)(F)F)cc(Cl)c2n1]

comprising:
(A2) contacting a compound of Formula 2 in a solvent Sα selected from toluene (PhCH₃), acetonitrile (MeCN), or combination thereof, with a compound of Formula 3 to form a compound of Formula 4:

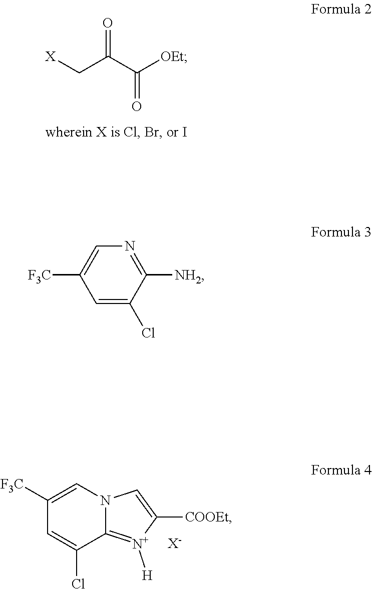

(B2) adding a base selected from ammonia, sodium hydroxide, potassium hydroxide, or mixtures thereof, in a solvent Sβ selected from water, methanol, ethanol, isopropanol, and mixtures thereof, to the reaction mixture (or isolated compounds of Formula 4) of Step (A2) to generate an intermediate of Formula 9:

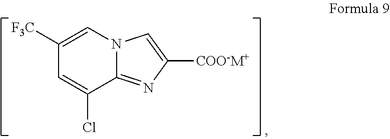

wherein M⁺ is an inorganic cation selected from sodium, potassium, lithium, and mixtures thereof; and
(C₂) adding an acid selected from hydrochloric acid (HCl), hydrobromic acid (HBr), phosphoric acid (H₃PO₄), sulfuric acid (H₂SO₄), or mixtures thereof, to the reaction mixture of Step (B2) to generate a compound of Formula 1.

In some embodiments, the solvent Sα and/or the solvent Sβ does not comprise ether for example 1,2-dimethoxyethane. In some embodiments, the solvent Sα and/or the solvent Sβ do not comprise tetrahydrofuran (THF).

In some embodiments, the methods provided herein reduce the amount at least one of the following impurities to less than 50 ppm:

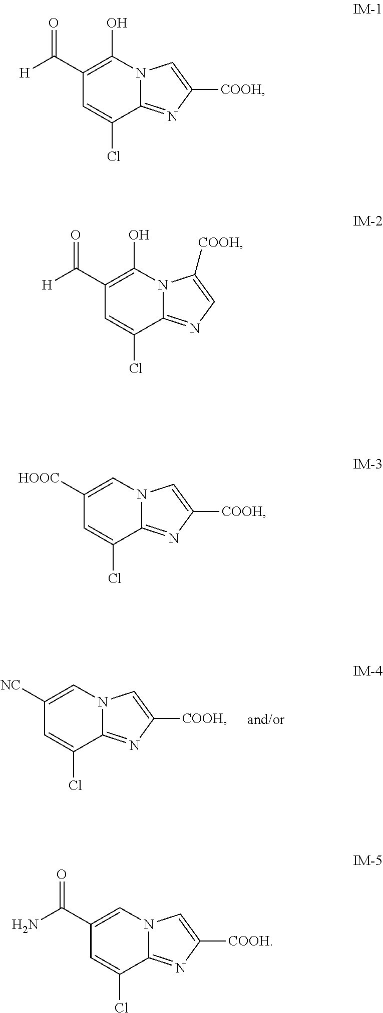 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1 ; N#Cc1cc(Cl)c2nc(C(=O)O)cn2c1 ; NC(=O)c1cc(Cl)c2nc(C(=O)O)cn2c1]

In some embodiments, the methods provided herein reduce the amount of at least one of the following impurities to less than 50 ppm:

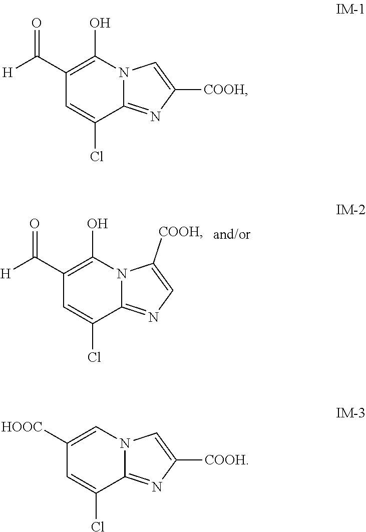 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1]

In some embodiments, the methods provided herein do not generate more than 50 ppm of any of the following impurities:

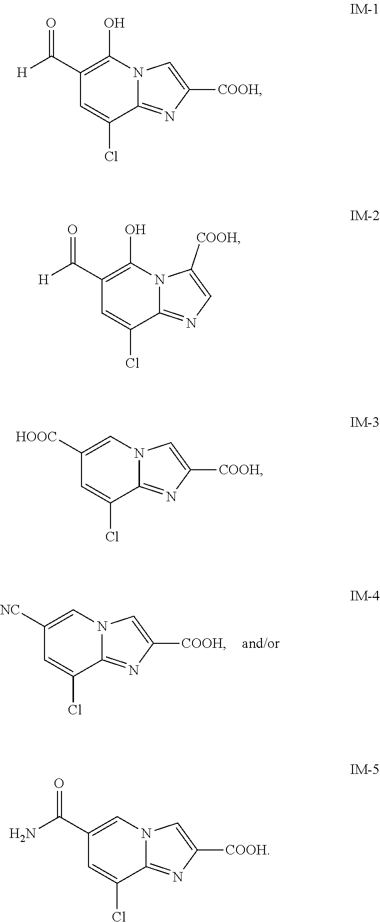 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1 ; N#Cc1cc(Cl)c2nc(C(=O)O)cn2c1 ; NC(=O)c1cc(Cl)c2nc(C(=O)O)cn2c1]

In some embodiments, the methods provided herein do not generate more than 50 ppm of any of the following impurities:

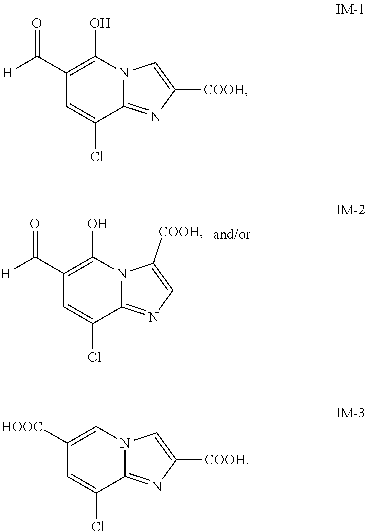 [SMILES: O=Cc1cc(Cl)c2nc(C(=O)O)cn2c1O ; O=Cc1cc(Cl)c2ncc(C(=O)O)n2c1O ; O=C(O)c1cc(Cl)c2nc(C(=O)O)cn2c1]

In another aspect, provided are intermediate compounds with a structure of Formula 9 and/or Formula 10:

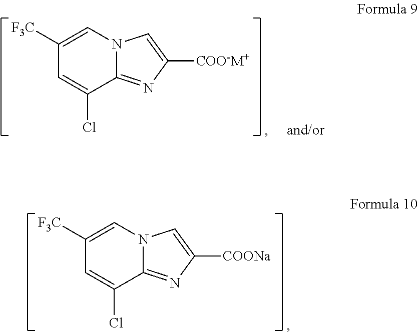

wherein M⁺ is an inorganic cation or organic cation.

In some embodiments, M⁺ is an inorganic cation selected from sodium, potassium, ammonium, lithium, and mixtures thereof. In some embodiments, M⁺ is sodium. In some embodiments, M⁺ is an organic cation selected from trimethylammonium, triethylammonium, tri-n-propylammonium, triisopropylammonium, and tributylammonium.

### DETAILED DESCRIPTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated.

For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such phrase would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

W here applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “ambient temperature” or “room temperature” as used in this disclosure refers to a temperature between about 18° C. and about 28° C.

In the above recitations, the term “alkyl”, includes straight-chain or branched alkyl, such as, methyl, ethyl, n-propyl, i-propyl, or the different butyl isomers. As used herein, haloalkanes are alkanes partially or fully substituted with halogen atoms (fluorine, chlorine, bromine or iodine). Examples of haloalkanes include CH₂Cl₂, ClCH₂CH₂Cl, ClCH₂CH₂CH₂CH₃, and CCl₃CH₃. Halogenated benzenes are benzenes partially or fully substituted with halogen atoms (fluorine, chlorine, bromine or iodine). Examples of halogenated benzenes include chlorobenzene, 1,2-dichlorobenzene and bromobenzene. C₇-C₁₀ aromatic hydrocarbons are compounds containing one benzene ring which is substituted with alkyl groups. Examples of C₇-C₁₀ aromatic hydrocarbons include toluene, xylenes, ethyl benzene and cumene (isopropylbenzene). C₅-C₁₀ aliphatic hydrocarbons are straight-chain or branched hydrocarbons. Examples of C₅-C₁₀ aliphatic hydrocarbons include n-hexane, mixed hexanes, n-heptane and mixed heptanes. C₅-C₁₀ cycloaliphatic hydrocarbons are cyclic hydrocarbons that can be substituted with straight-chain or branched alkyl groups. Examples of C₅-C₁₀ cycloaliphatic hydrocarbons include cyclopentane, methylcyclopentane, cyclohexane and methylcyclohexane.

As noted above, impurities may be generated during the synthesis of compounds of Formula D. Those impurities may cause a darker color of the products, and often re-crystalization is required to remove those impurities, which can be time comsuming, expensive, and/or decrease overall yield. Surprisingly by reversing the order of adding base and acid, or even adding acid alone (i.e., by adding the acid first or adding the acid alone), generation of such impurities can be eliminated or reduced into insignificant amount. In some aspects, the methods disclosed herein either eliminate at least one of those impurities, or do not produce any of those impurities at all, and thus are superior to the previous disclosed methods. The first aspect of the invention provides a method of preparing a compound of Formula D equal to Formula 1 where acid is added to compounds of Formula C equal to Formula 4 first.

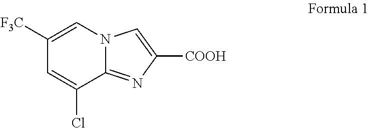 [SMILES: O=C(O)c1cn2cc(C(F)(F)F)cc(Cl)c2n1]

In some embodiments as shown in Scheme 1, compounds of Formula 2 can react with a compound of Formula 3 to form compunds of Formula 4, which can then be converted to compound of Formula 1 with an acid, or an acid follow by a base (for adjusting pH when needed).

In some embodiments as shown in Scheme 1, compounds of Formula 4 can be converted to a compound of Formula 1 with an acid, followed by a buffer, followed by a base (for adjusting pH when needed).

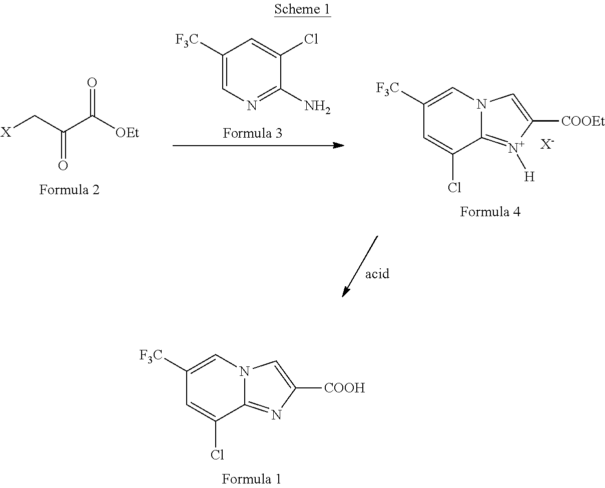

The cyclization and deesterification reactions shown in Scheme 1 can be conducted under a broad range of temperatures, i.e., temperatures in the range from 20° C. to 150° C.; or from 50° C. to 200° C. Temperatures in the range from 50° C. to 180° C.; or from 60° C. to 100° C. are particularly useful. Temperatures in the range of 60° C. to 95° C. are especially useful. Temperatures in the range of 75° C. to 95° C. are especially useful.

Suitable solvents include, but are not limited to, solvents such as benzene, toluene, xylene, chlorobenzene, dichlorobenzene.

A variety of mineral acids may be employed in the conversion of compounds of Formula 4 to Formula 1 (also compounds of Formula C to compounds of Formula D). Suitable mineral acids include, but are not limited to HF, HCl, HBr, HI, H₂SO₄, H₃PO₄, HNO₃, and HClO₄. Suitable concentrations of aqueous acid may be in the range of from about 10% to about 50%, from about 10% to about 40%, from about 15% to about 37%, from about 20% to about 30%. A range of molar equivalents, with respect to compounds of Formula 4 (Formula C), of acid may be employed. Examplary molar equivalents of acid include from about 1.5 to about 6.2 molar equivalents, from about 1.5 to about 5.0 molar equivalents, from about 1.5 to about 4 molar equivalents, from about 1.5 to about 3.1 molar equivalents.

The reaction temperature during during acid neutralization and pH adjustment may be in the range from about 5° C. to about 105° C., from about 10° C. to about 100° C., from about 15° C. to about 95° C., from about 20° C. to about 90° C., from about 25° C. to about 85° C., from about 30° C. to about 85° C., from about 35° C. to about 85° C., from about 40° C. to about 85° C., from about 45° C. to about 85° C., from about 50° C. to about 85° C., from about 55° C. to about 85 C, from about 60° C. to about 85° C., from about 65° C. to about 85° C., from about 70° C. to about 85° C., from about 75° C. to about 85° C., from about 75° C. to about 80° C.

A variety of buffers, having a pKa between 0 and 4, may be employed including, but not limited to, sodium acetate, sodium citrate, sodium oxalate, sodium malonate, trisodium phosphate, and sodium succinate. The buffers may be added to the reaction mixture as solids or as aqueous solutions. Aqueous buffer solutions may have a variety of concentrations for example from about 10 wt % to about 90 wt %, from about 10 wt % to about 80 wt %, from about 10 wt % to about 70 wt %, from about 10 wt % to about 60 wt %, from about 10 wt % to about 50 wt %, from about 10 wt % to about 40 wt %, from about 10 wt % to about 30 wt %.

A variety of bases may be employed. Bases may be added to the reaction mixture as solids or as aqueous solutions. A variety of aqueous basic solutions may be employed, including but not limited to, basic solutions made from LiOH, NaOH, KOH, CaO, MgO, Ca(OH)₂, Na₂CO₃, and K₂CO₃.

The final pH, after buffer addition and base addition may be in the range from about pH −1 to about pH 3.5, from about pH 0 to about pH 3.5, from about pH 1 to about pH 3.5, from about pH 1.5 to about pH 3.0, from about pH 1.75 to about pH 2.75, from about pH 2.0 to about pH 2.7, from about pH 2.2 to about pH 2.7.

The process shown in Scheme 1 is efficient and reduces the cost of production for the compound of Formula 1.

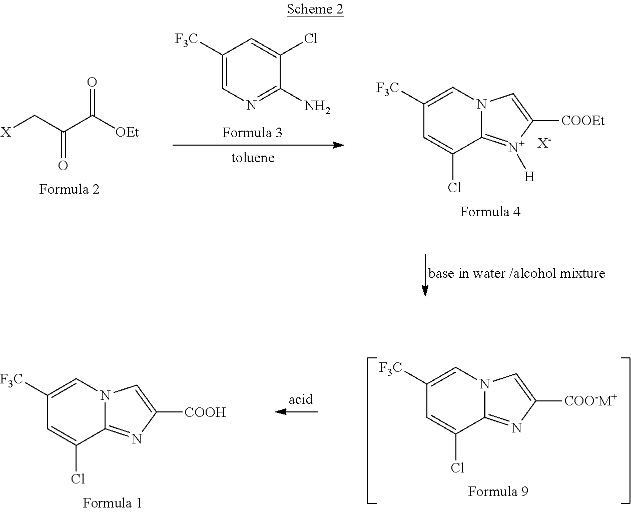

Another aspect of the invention provides a method of preparing a compound of Formula 1 where base is added to compounds of Formula 4 first. In some embodiments as shown in Scheme 2, compounds of Formula 2 can react with a compound of Formula 3 to form compounds of Formula 4 in a solvent containing toluene. A base in a solvent containing water/alcohol mixture is then added to the compound of Formula 4 to form intermediates of Formula 9, and then an acid is added to convert the intermediates of Formula 9 into compounds of Formula 1. The major differences as compared to the previously disclosed methods are the use of the solvent toluene first and then the base in a solvent containing water/alcohol mixture, where the intermediates of Formula 9 are created. Such differences produce unexpected results of eliminating or reducing undesired impurities as described herein.

Suitable temperature for the hydrolysis step can be between 0° C. and 60° C., preferably between 20° C. and 40° C. Suitable temperature for the acidification step can be between −10° C. and 60° C., preferably between 0° C. and 40° C.

### Preparation Example 1

A 250-mL jacketed reactor equipped with a twin blade agitator, a Dean-Stark trap, condenser, a programmable jacket heating bath, and an aqueous caustic scrubber is purged with N₂ for 30 minutes. A compound of Formula 2 (25.05 g crude, 79.5 wt %, 102 mmol, 1.18 equiv) is charged followed by toluene (PhCH₃; 101.46 g) and a compound of Formula 3 (17.04 g, 86.7 mmol, 1.0 equiv). The agitator is started at 400 rpm and a N₂ headspace sweep is started at 1-2 N₂ bubbles/second. The condenser is turned on and the Dean-Stark trap is filled with toluene (PhCH₃). The jacket bath is warmed to 110° C. and the reaction is heated for six and half hours and sampled for reaction completion by H PLC.

The vessel is cooled to 35° C. and agitation is increased to 600 rpm. Concentrated aqueous HCl (99.0 g solution, 35.5 wt %, 11 equiv) is charged over −30 seconds. The reaction changed from an orange slurry to an orange biphasic mixture with solids dissolved. The N₂ is switched from a sweep to an active pad. The reaction is stirred for three and half hours at 80° C. and sampled for completion by H PLC. The jacket bath is then cooled to 20° C. and the resulting orange slurry is stirred for an additional hour at 20-25 QC. The slurry is then vacuum filtered and collected on a Buchner. The vessel and product cake are washed with 2×50 g portions of fresh PhCH₃. The final product wet cake is further dried overnight in a vacuum oven (~40-60 torr, 100-105 QC) to afford an off-white solid compound of Formula 1 (18.72 g cake, 0.2 wt % H₂O, 18.68 g contained, 81% yield from compound of Formula 3).

### Preparation Example 2

To a jacketed reactor, water (40 g, 2 w/w) and compound of Formula 4 (X═Br) (20 g, 61.63 wt %) is charged, and the mixture is agitated at 35-45° C. IPA (40 g, 2 w/w) is charged into the mixture, followed by addition of aq. NaOH solution [prepared by dissolving NaOH (4.71 g, 2.27 eq.) in water (20 g, 1 w/w)] over one hour. Aq. HCl [prepared by dissolving HCl 36 wt %, 7.85 g, 1.54 eq.) in water (20 g, 1 w/w) is gradually charged over 30 minutes at 35-37° C. The reaction mixture is cooled to −10° C. and hold for 30 minutes. The resulting mixture is filtered and the solid is washed with water (20 g×2). The filter cake is dried at 103° C. and −0.096 M Pa vacuum overnight to afford 12.95 g compound of Formula 1 (99.5 wt %, 97.0% yield).

### Preparation Example 3

A 500 mL jacketed reactor equipped with a twin blade agitator, Dean-Stark trap, thermowell and a dip tube was purged with N₂ for 30 minutes. To the reactor was charged 39.84 g of compound of Formula 3 (1.0 equiv, 203 mmol), followed by 274.68 g of PhCH₃ and 50.45 g of compound of Formula 2 (X═Br) (1.16 equiv, 236 mmol). Agitation (400 rpm) was started followed by setting an N₂ headspace sweep at 1-2 bubbles/see, and the condenser was turned on. The Dean-Stark trap was filled with PhCH₃. The jacket bath was then set to 115° C. for 10 h. The reaction mixture changed from a yellow, hazy mixture to a dark orange slurry. The supernatant of the reaction was sampled for completion (measured 99.0% conversion of a compound of Formula 3) and considered complete. A few g of H₂O were observed in the Dean-Stark trap and removed.

The reaction was cooled to 35° C. and deionized H₂O (78.25 g) followed by 90.90 g of 37 wt % HCl(aq) were charged to give a yellow-orange slurry. The N₂ sweep was changed to a pad. The jacket bath was set to 105.0° C. and 92.0° C. (reflux) was achieved in the vessel after 35 minutes. After 4 h at a vessel T of 92-93° C., agitation was stopped and the lower aqueous layer was sampled for reaction completion via the dip tube. The reaction achieved 99.8% conversion and was deemed complete. The jacket temperature was lowered to 80.0° C. The collected aqueous distilled (38.54 g) was collected and analyzed for contained EtOH (140 mol % with respect to a compound of Formula 3) and pH<0. A solution of trisodium citrate dihydrate (22.31 g solid in 66.28 g deionized water) was prepared and loaded to a freshly equipped 250 mL addition funnel. The citrate solution was fed over 11 min at a vessel T=78.8-72.7° C.

The dip tube was then removed and replaced with a calibrated pH probe and meter. Caustic solution (95.93 g, 50 wt % NaOH in H₂O) was charged to the addition funnel. The solution was fed over 11 min at a vessel T range of 74.8-86.2° C. to a measured pH of 2.21 which was then held for 15 min and the pH was observed to change to 2.18. The neutralization was considered complete. Total caustic solution used was 68.15 g. The jacket T was then set to 20.0° C. and the mixture was stirred for 90 min until the vessel T reached 21.0 QC.

At vessel T=21.0° C., the pH was observed to be 2.09. The slurry was filtered via vacuum filtration and the vessel and cake were washed consecutively with 100 g of both PhCH₃ and deionized H₂O adjusted to pH 1.95. A wet cake of a compound of Formula 1 was then placed in a vacuum oven and dried overnight (~60-70 torr, 100-105° C., ~20 h). The resulting off-white, free-flowing solid (49.45 g) was then analyzed via LC and determined to be >99.9 wt. % of compound of Formula 1. Yield from a compound of Formula 3 was calculated to be 92.1%. The solid was stored at ambient lab temperature in a desiccator.

### Preparation Example 4

A 500 mL jacketed reactor equipped with a twin blade agitator, Dean-Stark trap, thermowell and a dip tube was purged with N₂ for 30 minutes. To the reactor was charged 70.02 g of compound of Formula 4 (X═Br) solid (50.4 g) followed by 249.01 g of PhCH₃. Agitation (300 rpm) was started followed by setting an N₂ pad at 1-2 bubbles/sec, and the condenser was turned on. The Dean-Stark trap was filled with PhCH₃. Deionized H₂O (71.14 g) and 83.71 g of 37 wt % HCl(aq) were charged to give a yellow-orange slurry.

The jacket T was set to 105.0° C. and 92.0° C. (reflux) was achieved in the vessel after 35 minutes. After 4 h at a vessel T of 92-93° C., agitation was stopped and the lower aqueous layer was sampled for reaction completion via the dip tube. The reaction achieved 99.9% conversion and was deemed complete. The jacket temperature was lowered to 80.0° C. The collected aqueous distilled (18.72 g) was collected and analyzed for contained EtOH (50.9 mol %) and pH<0. A solution of trisodium citrate dihydrate (20.23 g solid in 60.72 g deionized water) was prepared and loaded to a freshly equipped 250 mL addition funnel. The citrate solution was fed over 10 min at a vessel T=77.4-74.7° C.

The dip tube was then removed and replaced with a calibrated pH probe and meter. Caustic solution (76.26 g, 50 wt % NaOH in H₂O) was charged to the addition funnel. Agitation was increased to 400 rpm for better mixing. The solution was fed over 29 min at a vessel T range of 78.7-84.9° C. to a measured pH of 2.31 which was then held for 10 min and the pH was observed to change to 2.38. The neutralization was considered complete. Total caustic solution used was 73.48 g. The jacket T was then set to 20.0° C. and the mixture was stirred out overnight.

The next morning, the pH was observed to be 2.65 and the vessel temperature was 20.2° C. The slurry was filtered via vacuum filtration and the vessel and cake were washed consecutively with 91 g of both PhCH₃ and deionized H₂O. The wet cake of compound of Formula 1 was then placed in a vacuum oven and dried overnight (~60-70 torr, 100-105° C., ~20 h). The resulting off-white, free-flowing solid (46.47 g) was then analyzed via LC and determined to be 97.8 wt % compound of Formula 1. Yield from compound of Formula 4 (X═B r) was 92.0%. The solid was stored at ambient lab temperature in a desiccator.

### Preparation Example 5

A 500 mL jacketed reactor equipped with a twin blade agitator, thermowell and a dip tube was purged with N₂. To the reactor was charged 80.04 g of compound of Formula 4 (X═Br) (57.56 g) followed by 284.59 g of PhCH₃. Agitation (300 rpm) was started followed by setting an N₂ pad at 1-2 bubbles/second. Deionized H₂O (81.22 g) and 95.39 g of 37 wt % HCl(aq) were charged to give a yellow-orange slurry.

The jacket T was set to 85.0° C. and the vessel T achieved 82-83° C. after 30 minutes. After 8 h at a vessel T of 82-83° C., agitation was stopped and the lower aqueous layer was sampled for reaction completion via the dip tube. The reaction achieved 99.2% conversion and was deemed complete. The jacket temperature was lowered to 80.0° C. A solution of trisodium citrate dihydrate (23.05 g solid in 69.53 g deionized water) was prepared and loaded to a freshly equipped 250 mL addition funnel. The citrate solution was fed over 12 min at a vessel T=76.8-73.2° C.

The dip tube was then removed and replaced with a calibrated pH probe and meter. Caustic solution (92.39 g, 50 wt % NaOH in H₂O) was charged to the addition funnel. Agitation was increased to 400 rpm for better mixing. The caustic solution was fed over 30 min at a vessel T range of 74.8-84.3° C. to a measured pH of 2.35 which was then held for 25 min and the pH was observed to change to 2.39. The neutralization was considered complete. Total caustic solution used was 80.04 g. The jacket T was then set to 20.0° C. and the vessel achieved 22.4° C. after 90 min with a pH of 2.35.

The slurry was filtered via vacuum filtration and the vessel and cake were washed consecutively with 105 g of both PhCH₃ and deionized H₂O adjusted to pH 2.11. The wet cake of compound of Formula 1 was then placed in a vacuum oven and dried overnight (~60-70 torr, 100-105° C., ~20 h). The resulting white, free-flowing solid (55.37 g) was then analyzed via LC and determined to be 99.9 wt. % compound of Formula 1. Yield from compound of Formula 4 was 98.5%. The solid was stored at ambient lab temperature in a desiccator.

### Analysis Example 1

The products were analyzed by high-pressure liquid chromatography on an Agilent Zorbax SB-Phenyl column, 100×4.6 mm, 1.8 μm [P/N: 828975-912], at a column temperature of 40° C., and a flow rate of 1.0 mL/minute. An injection volume of 2 μL and a maximum pressure of 240 bar were employed. Two eluents A and B were used. Eluent A comprised water and 0.1% formic acid. Eluent B comprised acetonitrile. The following times and compositions were used in a gradient elution:

Gradient:

| Time (minutes) | % Eluent A | % Eluent B |
|---|---|---|
| 0 | 90 | 10 |
| 13 | 50 | 50 |
| 13.1 | 5 | 95 |
| 15 | 5 | 95 |
| 15.1 | 90 | 10 |

A post time of three minutes was employed, and methanol was used for needle wash. Peaks were observed with an ultraviolet detector at 250 nm. Peaks were analyzed with an Agilent Open Lab integrator. The following retention times were observed:

Retention Times*:

| | |
|---|---|
| IM -5 | 2.90 min |
| IM -3 | 3.55 min |
| IM -1 | 4.14 min |
| Internal Standard (Salicylamide) | 4.71 min |
| IM -4 | 5.13 min |
| IM -2 | 7.07 min |
| Formula 1 | 7.90 min |
| Formula 3 | 10.10 min |
| Formula 4 | 12.30 min |

What is claimed is:

1. A method of preparing a compound of Formula D: 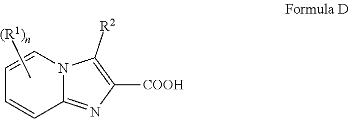
wherein each R¹ is independently halogen, nitro, SF₅, N(C₁-C₈ alkyl)(C₁-C₈ alkyl), C(═S)N(C₁-C₈ alkyl)(C₁-C₈ alkyl), SO₂N(C₁-C₈ alkyl)(C₁-C₈ alkyl), OSO₂(C₁-C₈ alkyl), OSO₂N(C₁-C₈ alkyl)(C₁-C₈ alkyl), N(C₁-C₈ alkyl)SO₂(C₁-C₈ alkyl), C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
n is 0, 1, 2, 3, or 4; and
R² is H, C₁-C₈ alkyl, C₁-C₈ haloalkyl, C₂-C₈ alkenyl, C₂-C₈ alkynyl, C₃-C₁₀ cycloalkyl, C₃-C₁₀ halocycloalkyl, C₄-C₁₀ alkylcycloalkyl, C₄-C₁₀ cycloalkylalkyl, C₆-C₁₄ cycloalkylcycloalkyl, C₅-C₁₀ alkylcycloalkylalkyl, C₃-C₈ cycloalkenyl, C₁-C₈ alkoxy, C₁-C₈ haloalkoxy, C₃-C₈ cycloalkoxy, C₃-C₈ halocycloalkoxy, C₄-C₁₀ cycloalkylalkoxy, C₂-C₈ alkenyloxy, C₂-C₈ alkynyloxy, C₁-C₈ alkylthio, C₁-C₈ alkylsulfinyl, C₁-C₈ alkylsulfonyl, C₃-C₈ cycloalkylthio, C₃-C₈ cycloalkylsulfinyl, C₃-C₈ cycloalkylsulfonyl, C₄-C₁₀ cycloalkylalkylthio, C₄-C₁₀ cycloalkylalkylsulfinyl, C₄-C₁₀ cycloalkylalkylsulfonyl, C₂-C₈ alkenylthio, C₂-C₈ alkenylsulfinyl, C₂-C₈ alkenylsulfonyl, C₂-C₈ alkynylthio, C₂-C₈ alkynylsulfinyl, C₂-C₈ alkynylsulfonyl, or phenyl;
comprising:
(b1) adding aqueous acid to a compound of Formula C to form a reaction mixture, wherein R³ is C₁-C₈ alkyl or C₁-C₈ haloalkyl, 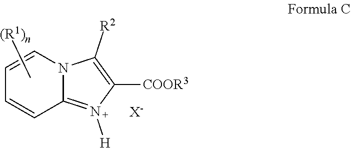
and;
(c1) adding base to the reaction mixture of Step (b1) to form a compound of Formula D.

2. The method of claim 1, wherein each R¹ is independently Cl, Br, I, C₁-C₆ alkyl, C₁-C₆ haloalkyl, C₁-C₆ alkoxy, C₁-C₆ haloalkoxy, or phenyl; and/or R² is H, C₁-C₆ alkyl, C₁-C₆ haloalkyl, C₁-C₆ alkoxy, or C₁-C₆ haloalkoxy.

3. The method of claim 1, wherein the acid used in Step (b1) is selected from hydrochloric acid (HCl), hydrobromic acid (HBr), phosphoric acid (H₃PO₄), sulfuric acid (H₂SO₄), or mixtures thereof.

4. The method of claim 1, further comprising Step (b2):
(b2) adding buffer to the reaction mixture of Step (b1) prior to Step (c1).

5. The method of claim 1, wherein the base used in Step (c1) is selected from sodium hydroxide, potassium hydroxide, or mixtures thereof.

6. The method of claim 4, wherein the buffer is selected from sodium acetate, sodium citrate, sodium malonate, sodium succinate, sodium triphosphate, or mixtures thereof.

7. The method of claim 4 for preparing a compound of Formula D, wherein a compound Formula D is Formula 1: 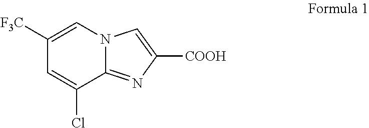 [SMILES: O=C(O)c1cn2cc(C(F)(F)F)cc(Cl)c2n1]
comprising:
(A1) adding aqueous acid selected from hydrochloric acid (HCl), hydrobromic acid (HBr), phosphoric acid (H₃PO₄), sulfuric acid (H₂SO₄), or mixtures thereof, to a compound of Formula C, wherein a compound of Formula C is Formula 4: 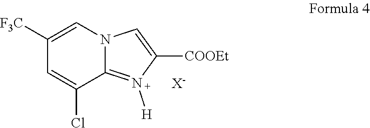
(B1) adding buffer selected from sodium acetate, sodium citrate, sodium malonate, sodium succinate, sodium phosphate, or mixtures thereof to the reaction mixture of Step A1; and
(C1) adding base selected from sodium hydroxide, potassium hydroxide, or mixtures thereof, to the reaction mixture of Step (B1).

8. The method of claim 2, wherein the acid used in Step (b1) is selected from hydrochloric acid (HCl), hydrobromic acid (HBr), phosphoric acid (H₃PO₄), sulfuric acid (H₂SO₄), or mixtures thereof.

9. The method of claim 2, further comprising Step (b2):
(b2) adding buffer to the reaction mixture of Step (b1) prior to Step (c1).

10. The method of claim 3, further comprising Step (b2):
(b2) adding buffer to the reaction mixture of Step (b1) prior to Step (c1).

11. The method of claim 8, further comprising Step (b2):
(b2) adding buffer to the reaction mixture of Step (b1) prior to Step (c1).

12. The method of claim 2, wherein the base used in Step (c1) is selected from sodium hydroxide, potassium hydroxide, or mixtures thereof.

13. The method of claim 3, wherein the base used in Step (c1) is selected from sodium hydroxide, potassium hydroxide, or mixtures thereof.

14. The method of claim 4, wherein the base used in Step (c1) is selected from sodium hydroxide, potassium hydroxide, or mixtures thereof.

15. The method of claim 9, wherein the base used in Step (c1) is selected from sodium hydroxide, potassium hydroxide, or mixtures thereof.

16. The method of claim 10, wherein the base used in Step (c1) is selected from sodium hydroxide, potassium hydroxide, or mixtures thereof.

17. The method of claim 11, wherein the base used in Step (c1) is selected from sodium hydroxide, potassium hydroxide, or mixtures thereof.

18. The method of claim 9, wherein the buffer is selected from sodium acetate, sodium citrate, sodium malonate, sodium succinate, sodium triphosphate, or mixtures thereof.

19. The method of claim 10, wherein the buffer is selected from sodium acetate, sodium citrate, sodium malonate, sodium succinate, sodium triphosphate, or mixtures thereof.

20. The method of claim 11, wherein the buffer is selected from sodium acetate, sodium citrate, sodium malonate, sodium succinate, sodium triphosphate, or mixtures thereof.

21. The method of claim 13, wherein the buffer is selected from sodium acetate, sodium citrate, sodium malonate, sodium succinate, sodium triphosphate, or mixtures thereof.

22. The method of claim 14, wherein the buffer is selected from sodium acetate, sodium citrate, sodium malonate, sodium succinate, sodium triphosphate, or mixtures thereof.

23. The method of claim 15, wherein the buffer is selected from sodium acetate, sodium citrate, sodium malonate, sodium succinate, sodium triphosphate, or mixtures thereof.

24. The method of claim 17, wherein the buffer is selected from sodium acetate, sodium citrate, sodium malonate, sodium succinate, sodium triphosphate, or mixtures thereof.
